(19) United States
(12) Patent Application Publication
(10) Pub. No.: US 2017/0027397 A1
(43) Pub. Date: Feb. 2, 2017

(54) Toilet Tab

(71) Applicant: James Robert Matthews, Anaheim, CA (US)

(72) Inventor: James Robert Matthews, Anaheim, CA (US)

(21) Appl. No.: 14/809,356

(22) Filed: Jul. 27, 2015

(51) Int. Cl. A47K 13/10; A61L 9/04; A47K 17/00

(52) U.S. Cl. CPC A47K 13/105; A47K 17/00; A61L 9/048

(57) ABSTRACT

A toilet seat lifting tab is disclosed. This tab is attached to the toilet seat to lift and place back the toilet seat easily and conveniently, without touching the toilet seat. The lifting tab is attached to the bottom of toilet seat with secured means, provided with defined spaces for including different designs and decors to present the toilet tab in a decorative and aesthetic manner. The invention is also provided with gel sanitizer and gel deodorizer to maintain the odor within the rest rooms.

### CROSS REFERENCE TO RELATED APPLICATION

Not Applicable

### FEDERALLY SPONSORED RESEARCH AND DEVELOPMENT Not Applicable

### MICROFICHE APPENDIX

Not Applicable

### BACKGROUND OF THE INVENTION

(1) Field of the Invention

The present invention relates to the field of the bathroom accessories. More particularly, the invention relates to a small tab that will be attached to the toilet seat for easy lifting up of the toilet seat without touching bottom of the seat.

(2) Background of the Invention

Users of toilet commodes, particularly at public locations, are oftentimes concerned about the possible spreading of disease by touching the toilet seat, which is obviously necessary in order to raise and lower said seat. The desire to avoid such manual touching has been previously addressed with only a limited degree of acceptability. Several previously proposed solutions have required an additional element to be physically attached to the toilet seat.

It is understood that a commonly encountered problem in the use of the conventional toilet or water closet is that the user must touch the seat in order to raise and lower it onto the toilet bowl. Toilet seats, and particularly the undersides thereof, due to their proximity to the bowl, tend to be unsanitary, and few people, if any, disinfect them after use. This concern is particularly acute as regards public toilets.

A number of structures have been devised to assist in the raising and the lowering of a conventional toilet seat. However, most of these prior structures cannot be readily affixed to the toilet seat without tools, making them generally unmanageable or difficult for those not adept with tools. The positions of these handles also cannot be readily changed after they are installed.

While devices mentioned in prior arts, fulfill their respective, particular objective and requirements, the aforementioned patents do not describe a hand-held toilet seat lifting device that allows a user to remotely and manually raise and lower a toilet seat.

In this respect, the hand-held toilet seat lifting device according to the present invention substantially departs from the conventional concepts and designs of the prior art, and in doing so provides an apparatus primarily developed for the purpose of allowing a user to remotely and manually grasp and lift or lower a toilet seat, thereby preventing the user's hand from being contaminated.

Therefore, it can be appreciated that there exists a continuing need for new and improved hand-held toilet seat lifting device which can be used for allowing a user to remotely and manually grasp and lift or lower a toilet seat, thereby preventing the user's hand from being contaminated. In this regard the present invention substantially fulfills this need.

### SUMMARY

In view of the foregoing disadvantages inherent in the known types of commode seat lifting apparatus present in the prior art, the present invention provides a new and improved commode seat lifting wherein the same provides a handle organization arranged for mounting to a commode seat and extending laterally thereof to provide a manual grasping arrangement for an associated commode seat spaced therefrom. As such, the general purpose of the present invention, which will be described subsequently in greater detail, is to provide a new and improved commode seat lifting apparatus which has all the advantages a the prior art commode seat lifting apparatus and none of the disadvantages.

An object of the invention is to provide an improved toilet seat lifting handle which is inexpensive to manufacture, which is inconspicuous in appearance when installed, which enables the user to easily raise a toilet seat without soiling his hands, and which can be cheaply manufactured by mass production methods.

It is an object of the invention to provide an improved toilet seat operating mechanism,

A definite advantage of the invention is its simplicity and efficiency. The invention is so efficient, simple and easy to use that persons without sight will have no difficulty in mastering its use and versatility.

Accordingly, it is an object of the present invention to provide a toilet seat handle assembly that may be easily and quickly repositioned.

It is another object of the present invention to provide a novel toilet seat handle assembly that may accommodate and adapt to a large range of toilet seat widths.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

To further clarify various aspects of some example embodiments of the present invention, a more particular description of the invention will be rendered by reference to specific embodiments thereof which are illustrated in the appended drawing. It is appreciated that the drawing depicts only illustrated embodiments of the invention and are therefore not to be considered limiting of its scope. The invention will be described and explained with additional specificity and detail through the use of the accompanying drawing in which:

FIG. 1 depict a perspective view of the present invention.

FIG. 2 A-B depict perspective view of the toilet tab with other accessories.

FIG. 3 is schematic representation of toilet tab with deodorizing option.

### DETAILED DESCRIPTION OF THE INVENTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

In some preferred embodiments, the present invention provides restroom accessories designed specifically to be used with toilet seats to allow the user to use the same with more hygiene and convenience. The purpose of the invention is to provide a user friendly, hygienic toilet tab or an accessory thereof.

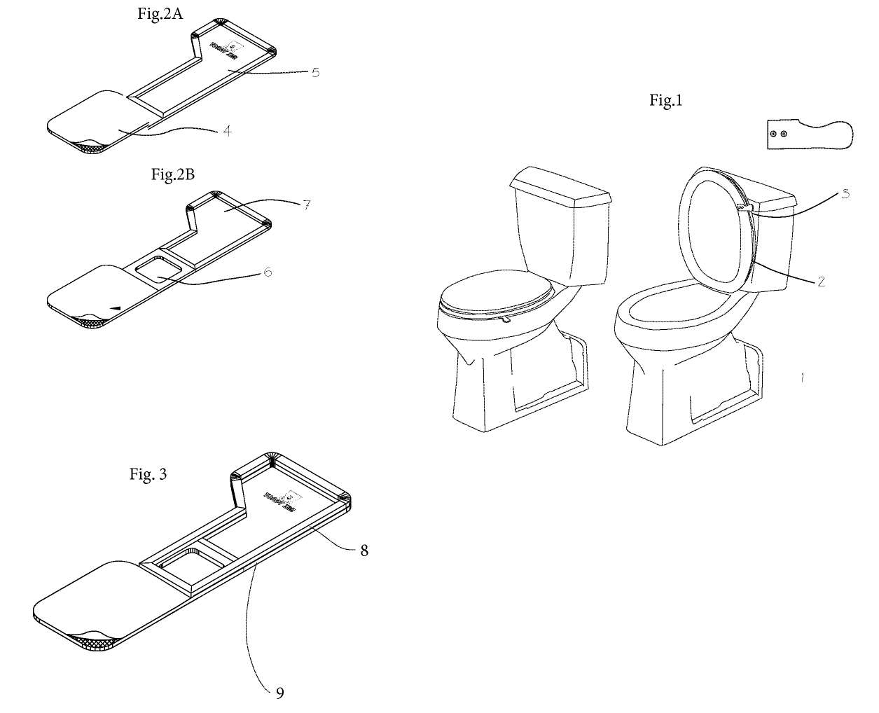

With reference to FIG. 1, the present invention is related to toilet 1 accessories. The toilet includes a toilet seat 2 that is lifted in order to use the toilet 1. Usually the toilet seats in public restrooms are not so hygienic to be lifted by the user's hand, and they end up getting in contact with germs and bacteria.

The present invention provides a toilet seat lifting tab 3 to be attached to toiler seat in a manner such that lifting of the toilet seat 2 before using the toilet 1 becomes more convenient. As seen in FIG. 2A, the toilet tab 3 comprises of a tab 4 with an additional is adjacent space 5 for placing logos and other decorative articles. In a preferred embodiment FIG. 2B, the toilet tab can also include defined space for gel deodorizer 6 arid gel sanitizer 7. As it shown on FIG. 3, the additional provision of gel deodorizer and gel sanitizer within the toilet tab also helps in maintain the odor within the restrooms, thus providing additional space by placing an additional panel 8 on the top of a base panel 9 for placing such odor maintaining agents and devices.

The present invention provides an inexpensive and simple device, having substantial universality of application, ease of use, and a high degree of sanitation. More specifically, a toilet seat lifting tab is attached to the underside of a toilet seat and a separate elongated wand is used to interact with said lifting tab. That is, the wand is not physically attached to the toilet seat. The wand has an elongated handle, with a manually graspable member at one end. The other end includes a hook that is manually engaged with the lifting tab. The wand is in one piece. This arrangement obviates the necessity of directly touching the seat. One can do without feeling uncomfortable under an assumption that the seat has been stained or soiled by others.

In a preferred embodiment of the present invention, it provides a tab attachment to be attached with the toilet seat for easy lifting up of the toilet seat without touching bottom of the seat that could be screwed in or stuck using double sided tape foam. The present invention can be used to attach public restrooms seat as well as permanent restrooms in home with an addition of gel tab deodorizer underneath. As disclosed above, the present invention uses possible cartoon characters imprinted on the tab attachment in order to make them more aesthetically appealing to kids. Different patterns or colors for compatibility for different decor in customer's restrooms could also be used.

It is purpose of the present invention to help people to keep the restrooms more sanitized and also aid younger men to have better restroom etiquette. This can be further used to teach kids better restroom using habits by utilizing the tab with different logos or fun characters displaying how to use restroom.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

I claim:

1. A toilet seat lifting tab comprising:
(a) a tab; and
(b) adjacent space for decors and logos.

2. The toilet seat lifting tab of claim 1, wherein said toilet tab attached to a toilet seat by screwing said tab with to the toilet seat.

3. The toilet seat lifting tab of claim 1, wherein said toilet tab attached to the toilet seat by double sided tape foam.

4. The toilet seat lifting tab of claim 1, wherein said toilet tab having adjacent space for decors or logos to present it aesthetically.

5. A toilet seat lifting tab comprising:
(c) a tab;
(d) adjacent space for decors and logos; and
(e) space for gel deodorize.

6. The toilet seat lifting tab of claim 5, wherein said toilet tab attached to a toilet seat by screwing said tab with to the toilet seat.

7. The toilet seat lifting tab of claim 5, wherein said toilet tab attached to the toilet seat by double sided tape foam.

8. The toilet seat lifting tab of claim 5, wherein said toilet tab having adjacent space for decors or logos to present it aesthetically.

9. The toilet tab as claimed in claim 5, wherein said space for gel deodorize having gel sanitizer and gel deodorizer to maintain the odor in the restrooms.
